SEQUENCE LISTING (22 sequences)

SEQ ID NO: 1 - DNA, 2175 bp, Nannochloropsis oceanica
  source 1..2175: mol_type = genomic DNA
  CDS 1..2172
atgggggcttctgtcttacgcagactggtgctgcttttactgccgctgcatggagcaactttcttcctttccagcagcagccagtatcatcgtcgctcaccaccaagcccgcagcaccatcgacactcttatccactgcacgccactaccgacgaaatactcaccgccaccgcccctaagcccacgcctgacctctgcatctttgatgctcccaaggccgttcgcctggccgccttttcctttgctacttacggagatcccactggctcccgatggctgcgtctgcccgacggcactgaccttggcttccaatctgatgacttggtgcgcaacaactttaaaagtttgctgttggttcacatcctgcaagcccgcaacctgccagaagaacggggcgggcttattgagagggctctctcaggctttagctgcgacccctacgtcatcacggccgtcaacaattcgtggggtgcagatatcgccatcactaaggccaagagtgctaattccaatcccgattttttccagcgctttttcttgtacgtggaagatcccaacacaacggatctagagctaactgtcaaggataagaacctgctgaaaggcgatgatgttattgggcactgtctggtgcccttgacgcagctgctcgacgggtcaaaaggagagggcgcacctggtcaagaaggcgtgatggagtgggcggggaccttgcaaatcgaaagcgtgcacaaggattttaaggatgctggcaccattgacgtagagctgcagctggtgcccttgaatggagaggccgtgaacaaggtcgaatacgactccaatgaccaggacgtcgccgagagcgacgccgatggcagtggcagcaagcatggtaaattcagtgcacggggtccgaaaagtgtgcttgaaggcattgattggaatgagctgggcgcccgtgtcggtggagtggccagggggctgggccgctaccagtttgcctgctttctgaccaacgaggagaccgatacgcaggggggaatctggtttgatgccaaagccaaggagatcattgtctccttccgaggcacgcagatggaatggaaggatttgctgactgacatggcgatctatcaagaagggttggatgggcctgaggacccgcggttggtgcacgcgggctttcgacgtgcttttcgttctattcaaggcggggtcctacaggccttgaattttatagcttccaatttggtggaggatggatggtctatagacgtgtgtgggcactccttagggggggcactggcgtgcttgatggcccacgagatcgatcggcgcatgcctgccttggtagaggaagggaggctgaatgttttttcctttggggcgccaagggtgggtaacacggcgttttgtgaggactatgacaagcggcttaaaaaccagagctttcgaatagtgaatgggttggacgttgttgcgcgaatgccacgagggtcgggtgaatcttccctattgatggaatacagtcattgcgggcgcacggtactactgaccgagacagaagacggtcgatttagtgtatgggtggagggcgaaagtgaaggcacttgcccactcaaagaaggagaggcatttacgagcacgacgcctacgagtcaatccatggcggagattatcaagaaggccgcaatagaagcgatggagagagggaagggaggtcttgagtttgaaactccatttagcaaggcaacctttgcgaacggtacagctgcctcagtgcaagaaatgctgcgggccgccggaaactttacaaaccttcaagctatgctctcctctacgtcctccatcgactacttctcctctttcacctccattccttcctcgccttctctggggccatttggacagctgagcttgcaaacggatttgcagcaaatcgccaatgcagtgtttggaagcgcgatgaactcgacagctacgattggcaagagcttggggttgaatgagacgtttgtagatagtgagttgagtttaattcgagggttggtgtacggagatgcattagcccaccatatggagccgctttattatgttgcgcttgagaaagcgtttgggattgcgatggaggaaggggtggatgacaagaaaggggaagagaagggggatggtgcaacgaagaggtacggataa

SEQ ID NO: 2 - protein, 724 aa, Nannochloropsis oceanica
MGASVLRRLVLLLLPLHGATFFLSSSSQYHRRSPPSPQHHRHSYPLHATTDEILTATAPKPTPDLCIFDAPKAVRLAAFSFATYGDPTGSRWLRLPDGTDLGFQSDDLVRNNFKSLLLVHILQARNLPEERGGLIERALSGFSCDPYVITAVNNSWGADIAITKAKSANSNPDFFQRFFLYVEDPNTTDLELTVKDKNLLKGDDVIGHCLVPLTQLLDGSKGEGAPGQEGVMEWAGTLQIESVHKDFKDAGTIDVELQLVPLNGEAVNKVEYDSNDQDVAESDADGSGSKHGKFSARGPKSVLEGIDWNELGARVGGVARGLGRYQFACFLTNEETDTQGGIWFDAKAKEIIVSFRGTQMEWKDLLTDMAIYQEGLDGPEDPRLVHAGFRRAFRSIQGGVLQALNFIASNLVEDGWSIDVCGHSLGGALACLMAHEIDRRMPALVEEGRLNVFSFGAPRVGNTAFCEDYDKRLKNQSFRIVNGLDVVARMPRGSGESSLLMEYSHCGRTVLLTETEDGRFSVWVEGESEGTCPLKEGEAFTSTTPTSQSMAEIIKKAAIEAMERGKGGLEFETPFSKATFANGTAASVQEMLRAAGNFTNLQAMLSSTSSIDYFSSFTSIPSSPSLGPFGQLSLQTDLQQIANAVFGSAMNSTATIGKSLGLNETFVDSELSLIRGLVYGDALAHHMEPLYYVALEKAFGIAMEEGVDDKKGEEKGDGATKRYG

SEQ ID NO: 3 - DNA, 2262 bp, Nannochloropsis oceanica
  source 1..2262: mol_type = genomic DNA
  CDS 1..2259
atgcccttgtgtcgggcgcgccagccgccgtggctgcgacagcgggcaccaattgtggttttgcttgtcctgcttttctgtttgctcaatcccgtcgtctcgtcatcactgcgggaaggacggcagcagcagcagcagcagcagcagtcgcaggaggtggaggagacaagaatacgtgaaaataccaagcaggagccggctgaggccatggtagagatcgatgcactgggccagcagcagcagcagcagcagcagcacgagcgagaggagcagttcacagcacaatcaccacccaagcccctccaacgcgaccgagaaggcgctggcattgcccgcctcctacaaagatggctagacgaagaggatggagcaacgaggcgagatcttcatgataatgaggaggagcataaagacaaggacatagaaaaagaggagaatgatgcgttagccttagctgacctaggcaacagcacaggcaacttacaggtatacaaagcccttcgggaaactgctgcggcccataatattacggcccgcgatttttggactttgcaacgtttgattttaacactgtcttttaatagatatgggaacaagccgaccatcttcccgatgtgtgaaatatataaaatagtggggcctttgcggttgcattgtgtcttcattgtctcgtcgtatcggttttacacggcgcagccgccgtcgttgaggaaggtggcgactgaacgctcgtacagtatttatagcggaggagagcaggaggagaagcagcagcaggagcaggagccaccacacgttttacaagacatcggtgtggtgcctgaattcatcgttgacaaacctttaattgagaaggaacggcctgtctatcgatggtgctggcggcagattgtaaatatgtatggctttacgtatcggtggaatgatgaaggagtagaagcggcgacgatggaacagcagcaacacacccaagagggagaaacaagggacaaagatggtggtgctcctccacctccctgcaatggtaaagagaacagcagcagcagtagcagcactgacagcgctcccctccttcctcccgaacctggctatgggaatcagagcctgctgtgggtggaggggccaatggacattccctttcgacggcccattggtcaagtatttacctacttccctcccccttgcagtagtagtagtggtactggttccagaaataggagtagcaggagtgatggtgaacaaaagtacgaggggagaggaggaggacatgtagagggaggaggtcctgagctggtggttgtgataagagggacattatacagtgaggaatggacggctaattttaggttgcagcgggcagatgaggatgaggtggagaagtatgattttgagggtattatggtaccgaagggtttttttgggttatttactgaagtacttccgcaggtcaatgatgcccttgtcctccacggcaacagccacatcactcggctgacactcaccggccattcactgggcgccagcattgcgcttctcctcgcggcatatttcggtcaaaaaagacccgacttaaaaatatccctcataacatttggtgggcccaacaccgtcgataacatattgtttgaaaactactggaaccatcaaataaatgtgcgccaagtgttttatatcggctcggggctcgtgcctcgggacgagagagacagatggttggagggagagagagaagagagacggcagcagcagcagcagtacgagtatcaggatcgtgaacaaccttctgattcccacttgagctttgggatcggggacatggttgcacagatgcccccagactgtactgctgtatccccctgtcccttgctcctcttccacgccaccaatggtctgacgaccaatgatgaccgcagtacaagtcccagtggcttcgatattgaaaacaatttcttcacgcgtctcaggctggcgcgtccgccggggtcgattgttttttatgcttacgacttaaagggacgtggctctgtgagcacatggttggcgtacgaagactttttgacgcatgtctggacaagattagatcgtggtgtaatttcaagtcatgtttgcgcgtacgcgtgttgggtatcttcgctgggaggtggaaagaatggcgaagaaacgcgatgttttttcccggaggaaggggagggacgaggcttgggggagatctgtgatttacaattgacgcgaggtgagcgtttgggaaaaacgcatctttaa

SEQ ID NO: 4 - protein, 753 aa, Nannochloropsis oceanica
MPLCRARQPPWLRQRAPIVVLLVLLFCLLNPVVSSSLREGRQQQQQQQQSQEVEETRIRENTKQEPAEAMVEIDALGQQQQQQQQHEREEQFTAQSPPKPLQRDREGAGIARLLQRWLDEEDGATRRDLHDNEEEHKDKDIEKEENDALALADLGNSTGNLQVYKALRETAAAHNITARDFWTLQRLILTLSFNRYGNKPTIFPMCEIYKIVGPLRLHCVFIVSSYRFYTAQPPSLRKVATERSYSIYSGGEQEEKQQQEQEPPHVLQDIGVVPEFIVDKPLIEKERPVYRWCWRQIVNMYGFTYRWNDEGVEAATMEQQQHTQEGETRDKDGGAPPPPCNGKENSSSSSSTDSAPLLPPEPGYGNQSLLWVEGPMDIPFRRPIGQVFTYFPPPCSSSSGTGSRNRSSRSDGEQKYEGRGGGHVEGGGPELVVVIRGTLYSEEWTANFRLQRADEDEVEKYDFEGIMVPKGFFGLFTEVLPQVNDALVLHGNSHITRLTLTGHSLGASIALLLAAYFGQKRPDLKISLITFGGPNTVDNILFENYWNHQINVRQVFYIGSGLVPRDERDRWLEGEREERRQQQQQYEYQDREQPSDSHLSFGIGDMVAQMPPDCTAVSPCPLLLFHATNGLTTNDDRSTSPSGFDIENNFFTRLRLARPPGSIVFYAYDLKGRGSVSTWLAYEDFLTHVWTRLDRGVISSHVCAYACWVSSLGGGKNGEETRCFFPEEGEGRGLGEICDLQLTRGERLGKTHL

SEQ ID NO: 5 - DNA, 1461 bp, Nannochloropsis oceanica
  source 1..1461: mol_type = genomic DNA
  CDS 1..1458
atgaccctaaccatcagcagcaggccaggtagagcgtcgcgggcctcgaggagaagacggctctgcttcgcggccacgctcgtgatgagcgccgtcggtggcacagcggccctacaccaaccacaccgcccttttgatgccgaggttacgcccgaagggggaactcgggagctcaaactcgacgaggccctggcgggagagtggggtttccggcttctggagccattcatggtctacaatgaaagtcatgtcacgccccagaatcatgacccatggtttccgggctgccctgtctttgacaacctcaacataaacgcccactttggctgccactttatccgactctgcaaagatgtcttgactgctttttgggtagattttagcaagactgccagtaccacgcgacccctgaacccgaacggtctcctgccgcagatcctcgccgataatcgaccgaatccaccgcgccaagtggaccgaactaccgtctacgccgagcgccccacttactactactgctaccgactgatcttactcatgtactctattgcctttgaatacgagaggagcaacatgcaatatagcctgccaagactcgtagacgactgggatcaggagcgctggtacatggtcaattatctcaaaccgcgaaacatcaaccggattgagcttaagggacgccagtcacctatagcagtttccatgatgaaaggtgaccggatgatgattgtgattcgtggtacgatttggacggaggagtggttcaaggactttcagtacaagtgggcgtatgatccggagaacctgttcccaggcaagacacacgaaggcatgtactctgtattcatcgatttttataagggactggaggaagaagtcgaattaaaaaagcccacacacgtcttggtaacgggccacagcttaggggcaggattggcgaatatgatcgggttgcgattggagaacaacatgcaacacaatatgcgcgtggacgtggtgggatttgggggccccaacactggagacatcgagtttgccgaggtgttccgcaagaccgtcaatagccgacatattattttcctaggtcgcgggaatgagccgcactcgcacatgtacacggcaggcgacattactgcgcaatacacctgcgagccttatcctggatgcgacatcctgtccacgggtccctttgggtatttctaccattacattcgcccgcacaaccaagtccccttctatgccgaagaaatgccgaatccaaagaagtggaacgaagtctccaacattaggcgcatcattccttcgaccaagggggtgattgcttcgcatctatgtagttacatttgctggacttctcaggctgtcggggatttggacagccggtgctattttgcaaatgagccgggccggcctgacttgccccaatcctactattgcgacgtgagtcaggttcccatatag

SEQ ID NO: 6 - protein, 486 aa, Nannochloropsis oceanica
MTLTISSRPGRASRASRRRRLCFAATLVMSAVGGTAALHQPHRPFDAEVTPEGGTRELKLDEALAGEWGFRLLEPFMVYNESHVTPQNHDPWFPGCPVFDNLNINAHFGCHFIRLCKDVLTAFWVDFSKTASTTRPLNPNGLLPQILADNRPNPPRQVDRTTVYAERPTYYYCYRLILLMYSIAFEYERSNMQYSLPRLVDDWDQERWYMVNYLKPRNINRIELKGRQSPIAVSMMKGDRMMIVIRGTIWTEEWFKDFQYKWAYDPENLFPGKTHEGMYSVFIDFYKGLEEEVELKKPTHVLVTGHSLGAGLANMIGLRLENNMQHNMRVDVVGFGGPNTGDIEFAEVFRKTVNSRHIIFLGRGNEPHSHMYTAGDITAQYTCEPYPGCDILSTGPFGYFYHYIRPHNQVPFYAEEMPNPKKWNEVSNIRRIIPSTKGVIASHLCSYICWTSQAVGDLDSRCYFANEPGRPDLPQSYYCDVSQVPI

SEQ ID NO: 7 - DNA, 2094 bp, Nannochloropsis oceanica
  source 1..2094: mol_type = genomic DNA
  CDS 1..2091
atgcggcaacgcaggcggaggctgtcggcggtgtttgcggccggcagcctgacgttgctggtcttgttgagcatgggccaaggagctattgccgacatcgccgccggtgccgggcagactggcagatccgcccacaccacgaaaatagccttaatggaaatgcctgattctgtaggtccgctgaggagcaaggacaagatcatcccctcggtggcattggaggatgtgtggatagagagggagaaagaggaagaggatgaggaagttcatgtcgaaggaaacctaattcccgcgccattaccggcgaaagccacgagcgaaaaaaagttgcgacagctggatagacaagagaagactactacttcatctcctcccaccaaggccgccacagcaaccgtaggagcggcagcccctctagacccagcagtcgttgttcccaccaaggaaacccgtatattaccctgggagacgaaagcatggcaacgagatcttgacgaactcaatgccttgcttgacatggacattgaacctgccgccattaacacagaggcctcatccctcgctgccgagattgggagtgacgggcactttgagactatgggtagtatgggaaatcttatgaacctcctctccccctcaccagaccctgcttggcctttctgctcccctgttggcatcctgtataaaacaggattggtttgcccggtcatgaatattgcggccaagattctgatgaggtctcccccggactttgagaaattgacaacggagcatcattgtcctaaccctactggggagctcccagaacagttggaggagctgagggaaggtggtttgccgatgccgatgattattcccaaggaggtggtggagagggagaagcctacatatgcgtggtatttgacgcatgtttacaatatgtacgcctttgccttctcctacgcggacgacatcctcagcacccacagcaacgcagactaccacacgctccgccccctaaagcccaagggatgggagaaccaaaacgtgtacttcgttaaggaccccgaaggttggaattggcccatggggcagtcactcacgaaggggaatagcatggtggtcttcttctgcggaaccatgttcccgggcggatgggaggcagactttaagacgagctttgcggcgaaatcggagacggagaagacgggtttgccgggccgagtgcatgatggctttttcgacttcgcccagcagtttttcgaggatttcacccgacaggtcgatctttacggtattcaaagcgtgacattcgctggacacagcctgggcggagctatctcacaggtggccgcggccttcatggtcaagcattacagtcacatcaaaattaattgcgtcacattcggatcaccacaggtcggcgatcggaccttcactgatttcttgaacgcccgtgtcaatctccgacgcatggcctacattggatccgggcagcttaatgagccggcagaacgaggccttaattacggcctgggcgacgtgatcactcagatgcctctggcgtgctacaacctctccatgcccttttgctcgcatatgacgtttatcgacaacaaggacgatctctcaagtttccaattcgtcatggcggcgggcaacgttccgatctacgcttccgacctgcgaaatacccaggagtggctgaaaaaggaaaattttctttttggagcccggacgtttagagacatcccagaaggagcaatggcttgtcacatatgctcctatgcgtgctggacctcggccgcaactgacgaccctgacaaccggtgttatttctccgggaagggagggagggagggagggaaggagggagggcaggcggaagggaggtatttgtgcgagattgatttgaccgagagtttgactgctcgttttcccaatttggaagggaatccggttaatagcgacttggtgcgtgaggggtcagtggcagaggggaagggagggggaggaggaggaggaggaggaggaggaggaggaggaggcggaggaggcggcgggttaatggatggaatgttgatgggggggaaaagggggttgaggatcaagtag

SEQ ID NO: 8 - protein, 697 aa, Nannochloropsis oceanica
MRQRRRRLSAVFAAGSLTLLVLLSMGQGAIADIAAGAGQTGRSAHTTKIALMEMPDSVGPLRSKDKIIPSVALEDVWIEREKEEEDEEVHVEGNLIPAPLPAKATSEKKLRQLDRQEKTTTSSPPTKAATATVGAAAPLDPAVVVPTKETRILPWETKAWQRDLDELNALLDMDIEPAAINTEASSLAAEIGSDGHFETMGSMGNLMNLLSPSPDPAWPFCSPVGILYKTGLVCPVMNIAAKILMRSPPDFEKLTTEHHCPNPTGELPEQLEELREGGLPMPMIIPKEVVEREKPTYAWYLTHVYNMYAFAFSYADDILSTHSNADYHTLRPLKPKGWENQNVYFVKDPEGWNWPMGQSLTKGNSMVVFFCGTMFPGGWEADFKTSFAAKSETEKTGLPGRVHDGFFDFAQQFFEDFTRQVDLYGIQSVTFAGHSLGGAISQVAAAFMVKHYSHIKINCVTFGSPQVGDRTFTDFLNARVNLRRMAYIGSGQLNEPAERGLNYGLGDVITQMPLACYNLSMPFCSHMTFIDNKDDLSSFQFVMAAGNVPIYASDLRNTQEWLKKENFLFGARTFRDIPEGAMACHICSYACWTSAATDDPDNRCYFSGKGGREGGKEGGQAEGRYLCEIDLTESLTARFPNLEGNPVNSDLVREGSVAEGKGGGGGGGGGGGGGGGGGGGGLMDGMLMGGKRGLRIK

SEQ ID NO: 9 - DNA, 2286 bp, Nannochloropsis oceanica
  source 1..2286: mol_type = genomic DNA
  CDS 1..2283
atgaagtttggtctgtatctgcgcgagaacgtggtggaagaatggaagcaatactacttgcaatacgacaaactaaagcgaatgattcgcattctggccgaagtggagagcaaagccatggcccccgaacctaccttgacgggcaaggttggcttttcgcttacggttcctccgcctaccaatgcagcggcgcagcctctcggccgtgcgggcaagggcatgggaggcgtgatggacggagactcggatgatgaagattcctcaggcggcgacgaggcggacgctgcgcccgtgacgcacgagttatttttcgatctgttggagaaggaaattcagaaggtacacaacttcaccgacaggaaagtgactgaaattagggccaaattgcgggatgtgggaaagagactgaaaattggcacgatagaagtcgtgcccggggggcagcctttggagataggggatgctgtgcggaaggaggtggacgaggtgggcgagcaatttttgcggttggaaaagtacgtgaacctaaattacactgggtttcacaagattttgaagaagcatgacaggtggttgacgaacccgtgtcggactttttatttgcagaggctgcagaaccataattggacgcaaggcgattattcggatgtggtggtgacgatgagccagatctggtcggcgttgaggggggatgttgctcctgaggggaccgcgacggagagtcaggagttcgtgcggagcactaccaagtactggattcaagacgaggatatttctcagttgaagtggtttgtgctgcagcacttgccggtgttgctccaagagagcatgggaaccaaatccgattctcagttggtgaattcggtgtacttggacaatgccacgttagagctgtataaaggacggttggacaagacaccaggagcgatcgcggttcgattcaggtggtatgggtccggtactcccgagcttgtttttgtggagcggaagacccaccgcgaggcatggactgcggagatgagcgtgaaagaacgcttcacggtgcatccttcggaagtaccggagattttggcgggcaggttcgacaaggccaagcatgtggagaagatgcgggcgaaggggaaaagcgagaaggaggtggaggattgggacatcttggtgacggaggtgtgccaggcgattaacagcaagcagcttgtcccgacgctgaggacgcagtacatgcggacggcctttcaaattccgttcgatgccacggtgcggatcagcctggacacgaatctgtgcatgctgacggaaacggggcggttgggcatggaccaggacaggtggttccgagacccgagcaagccggtgccgcggaatgagatcacccgctttccccacgccgtgcttgaggtgaagctgcaattaaaggacgagggggccaagccgcagtgggtcacggatttgttgagttcaggcatcccccgcgaagtccacaagttttctaagtttatccacggatgcgccgtcctgctgccagaggaagtgcaggccatgccctactggatcgacgacccctcattgcgcgagtccattgctgcctcggggtccgagaatatcttggaaccggagagtgggaagaagggaggggtgggaggaatgctggcgcacatgctgccgcatgggaaagaagggaaggagaaggcgaaaacgactgcgctgtctgcagcagcaaaacgaacgcccgccccgacaccaaggagggacagcgagacaccgcttccttcaagggtgtcagtgggtgcaagtaaaggaaaggcatggtcggcgcctttgatggcctccacggaggaggagtattatgaggacgaggagggcgggcctggttgttgggagagtatgtgtggagggtgtttcgcctgggcagctccacccgagatttcggcacggatggcgcagcagaaagtggagcccaagctgttctttgcgaatgaacggacatttatccattggttgaatatggccgtgacgatttcttccctgggcgcggcggtcttggccttctcccccgaggacaccgtgagtgagctgtacggaattatcttgatgcccgtgtcgttgttattcgctttgtacgccctcaacacctacatcacgcgaagcgcgaagatccggacgcgggagccgacgaggtgggatgacccgatggggcccgtgcttttggggagtatcttcacattggcgttgacagctcagtttttgattaagttggcccatgtgttgaagcaggatgaggtctag

SEQ ID NO: 10 - protein, 761 aa, Nannochloropsis oceanica
MKFGLYLRENVVEEWKQYYLQYDKLKRMIRILAEVESKAMAPEPTLTGKVGFSLTVPPPTNAAAQPLGRAGKGMGGVMDGDSDDEDSSGGDEADAAPVTHELFFDLLEKEIQKVHNFTDRKVTEIRAKLRDVGKRLKIGTIEVVPGGQPLEIGDAVRKEVDEVGEQFLRLEKYVNLNYTGFHKILKKHDRWLTNPCRTFYLQRLQNHNWTQGDYSDVVVTMSQIWSALRGDVAPEGTATESQEFVRSTTKYWIQDEDISQLKWFVLQHLPVLLQESMGTKSDSQLVNSVYLDNATLELYKGRLDKTPGAIAVRFRWYGSGTPELVFVERKTHREAWTAEMSVKERFTVHPSEVPEILAGRFDKAKHVEKMRAKGKSEKEVEDWDILVTEVCQAINSKQLVPTLRTQYMRTAFQIPFDATVRISLDTNLCMLTETGRLGMDQDRWFRDPSKPVPRNEITRFPHAVLEVKLQLKDEGAKPQWVTDLLSSGIPREVHKFSKFIHGCAVLLPEEVQAMPYWIDDPSLRESIAASGSENILEPESGKKGGVGGMLAHMLPHGKEGKEKAKTTALSAAAKRTPAPTPRRDSETPLPSRVSVGASKGKAWSAPLMASTEEEYYEDEEGGPGCWESMCGGCFAWAAPPEISARMAQQKVEPKLFFANERTFIHWLNMAVTISSLGAAVLAFSPEDTVSELYGIILMPVSLLFALYALNTYITRSAKIRTREPTRWDDPMGPVLLGSIFTLALTAQFLIKLAHVLKQDEV

SEQ ID NO: 11 - DNA, 18 bp, synthetic construct
  misc_feature 1..18: note = Description of Artificial Sequence: TALEN target sequence
  source 1..18: mol_type = other DNA
tgctagaagccgcaccgc

SEQ ID NO: 12 - DNA, 15 bp, synthetic construct
  misc_feature 1..15: note = Description of Artificial Sequence: spacer
  source 1..15: mol_type = other DNA
tagcatcatgaagtt

SEQ ID NO: 13 - DNA, 18 bp, synthetic construct
  misc_feature 1..18: note = Description of Artificial Sequence: TALEN target sequence
  source 1..18: mol_type = other DNA
tggtctgtatctgcgcga

SEQ ID NO: 14 - DNA, 18 bp, synthetic construct
  misc_feature 1..18: note = Description of Artificial Sequence: TALEN target sequence
  source 1..18: mol_type = other DNA
tgcaatacgacaaactaa

SEQ ID NO: 15 - DNA, 15 bp, synthetic construct
  misc_feature 1..15: note = Description of Artificial Sequence: spacer
  source 1..15: mol_type = other DNA
agcgaatgattcgca

SEQ ID NO: 16 - DNA, 18 bp, synthetic construct
  misc_feature 1..18: note = Description of Artificial Sequence: TALEN target sequence
  source 1..18: mol_type = other DNA
ttctggccgaagtggaga

SEQ ID NO: 17 - DNA, 18 bp, synthetic construct
  misc_feature 1..18: note = Description of Artificial Sequence: TALEN target sequence
  source 1..18: mol_type = other DNA
ttccagcagcagccagta

SEQ ID NO: 18 - DNA, 15 bp, synthetic construct
  misc_feature 1..15: note = Description of Artificial Sequence: spacer
  source 1..15: mol_type = other DNA
tcatcgtcgctcacc

SEQ ID NO: 19 - DNA, 18 bp, synthetic construct
  misc_feature 1..18: note = Description of Artificial Sequence: TALEN target sequence
  source 1..18: mol_type = other DNA
accaagcccgcagcacca

SEQ ID NO: 20 - DNA, 18 bp, synthetic construct
  misc_feature 1..18: note = Description of Artificial Sequence: TALEN target sequence
  source 1..18: mol_type = other DNA
tcagtctgcggcatgccc

SEQ ID NO: 21 - DNA, 15 bp, synthetic construct
  misc_feature 1..15: note = Description of Artificial Sequence: spacer
  source 1..15: mol_type = other DNA
ttgtgtcgggcgcgc

SEQ ID NO: 22 - DNA, 18 bp, synthetic construct
  misc_feature 1..18: note = Description of Artificial Sequence: TALEN target sequence
  source 1..18: mol_type = other DNA
cagccgccgtggctgcga

(19) United States
(12) Patent Application Publication
(10) Pub. No.: US 2026/0167921 A1
(43) Pub. Date: Jun. 18, 2026

(54) ALGAE WITH MODIFIED LIPASE GENE AND SPX2 GENE, AND METHOD OF PRODUCING TRIACYLGLYCEROL USING THE SAME

(71) Applicants: PHYTOLIPID TECHNOLOGIES CO., LTD., Yokohama-shi, Kanagawa (JP); HIROSHIMA UNIVERSITY, Higashihiroshima-shi, Hiroshima (JP); MAZDA MOTOR CORPORATION, Aki-gun, Hiroshima (JP)

(72) Inventors: Hiroyuki OHTA, Tokyo (JP); Mie SHIMOJIMA, Tokyo (JP); Masako IWAI, Yokohama-shi (JP); Atsushi SAKAMOTO, Higashihiroshima-shi (JP); Takashi YAMAMOTO, Higashihiroshima-shi (JP); Kumiko OKAZAKI, Higashihiroshima-shi (JP); Tomokazu KURITA, Higashihiroshima-shi (JP); Shinichiro MAEDA, Aki-gun (JP)

(73) Assignees: PHYTOLIPID TECHNOLOGIES CO., LTD., Yokohama-shi, Kanagawa (JP); HIROSHIMA UNIVERSITY, Higashihiroshima-shi, Hiroshima (JP); MAZDA MOTOR CORPORATION, Aki-gun, Hiroshima (JP)

(21) Appl. No.: 19/414,618

(22) Filed: Dec. 10, 2025

(30) Foreign Application Priority Data
Dec. 13, 2024 (JP) 2024-219013

(51) Int. Cl. C12N 1/12; C12N 9/20; C12N 15/79; C12P 7/6445; C12R 1/89

(52) U.S. Cl. CPC C12N 1/12; C12N 9/20; C12N 15/79; C12P 7/6445; C12R 2001/89; C12Y 301/01003

(57) ABSTRACT

Provided is means for achieving increase in the amounts of triacylglycerol accumulated in algae with high lipid accumulation. An alga having the following characteristics (1) and (2): (1) having reduced expression of a class 3 lipase gene; and (2) having reduced expression of an SPX2 gene.

### REFERENCE TO ELECTRONIC SEQUENCE LISTING

The application contains a Sequence Listing which has been submitted electronically in .XML format and is hereby incorporated by reference in its entirety. Said .XML copy, created on Dec. 5, 2025, is named “FP-350 sequence.xml” and is 39,494 bytes in size. The sequence listing contained in this .XML file is part of the specification and is hereby incorporated by reference herein in its entirety.

### TECHNICAL FIELD

The present invention relates to an alga that accumulates a large amount of triacylglycerol, and a method of producing triacylglycerol using the same.

### BACKGROUND ART

The biomass of algae is larger than that of plants, and algae are advantageous in biofuel production and useful-lipid production (Non Patent Literature 1). Research is globally ongoing to produce industrial and food products including biofuels from biomass such as alga-derived triacylglycerol (TAG) as a raw material. However, the current production cost is disadvantageously high for the progress of commercialization. Accordingly, further technical development is essential for production cost reduction.

Reduction in nutrient salts causes growth reduction and accumulation of lipid such as TAG in most plants and algae. Some SPX proteins, each having an SPX domain, are known to be involved in absorption, transport, and storage of inorganic phosphate, and signaling to control them (Non Patent Literature 2). The eustigmatophyte Nannochloropsis (hereinafter, referred to as “Nannochloropsis”) is an ultra-microalga, and reported to accumulate a certain amount of TAG in normal culture, and accumulate a larger amount of TAG under nutrient-deficient conditions, wherein the lipid composition is simple and suitable for fuels (Non Patent Literature 1, Non Patent Literature 3). Nannochloropsis allows seawater culture and high-density culture, thus enabling low-cost production, and is characterized in that gene modification techniques have been already established therefor (Non Patent Literature 4, Non Patent Literature 5). Previously, the present inventors have discovered that the loss of the function of SPX2, which responds to phosphorus deficiency, in Nannochloropsis results in increase in accumulation of triacylglycerol (Patent Literature 1).

The amount of lipid accumulated is regulated through lipid degradation and lipid synthesis. Lipase is known as a protein involved in lipid degradation. Both TGL and SDP1, major TAG lipases known in budding yeasts and higher plants, have a Patatin like domain (Non Patent Literature 6, Non Patent Literature 7, Non Patent Literature 8). It has been reported that if the expression level of an AtSDP1 homolog gene in a diatom (Phaeodactylum tricornutum) is reduced to 20% to 40% thereof by means of RNAi, the amount of lipid doubles (Non Patent Literature 9). Nannochloropsis has lipases of the same domain structure, TGL1 and TGL2. A TGL1/TGL2 double mutant has been produced, and found to exhibit increase in the amount of lipid accumulated on day 2 in the early stage of culture (Non Patent Literature 10). However, the increase in the amount of TAG accumulated was not found in the late stage of culture or under nutrient-deficient conditions, and hence the TAG degradation in Nannochloropsis is expected to be mainly caused by an unknown lipase localized in lipid droplets other than TGL1 and TGL2, which has not been identified yet.

### CITATION LIST

### Patent Literature

[Patent Literature 1] International Publication No. WO 2020/050412

### Non Patent Literature

[Non Patent Literature 1] Plant J. 54, 621-639, (2008)
[Non Patent Literature 2] Liu et al., Open Biol. 2018-Jan 3; 8 (1): 170231
[Non Patent Literature 3] Biotechnol. Bioeng. 102, 100-112, (2009)
[Non Patent Literature 4] Proc. Natl. Acad. Sci. USA 108, 21265-21269, (2011)
[Non Patent Literature 5] Genes Cells 25, 695-702, (2020)
[Non Patent Literature 6] Plant Cell 18, 665-675, (2006)
[Non Patent Literature 7] J. Biol. Chem. 278, 23317-23323, (2003)
[Non Patent Literature 8] J. Biol. Chem. 280, 37301-37309, (2005)
[Non Patent Literature 9] Biochim. Biophys. Acta 1861, 239-248, (2016)
[Non Patent Literature 10] Biochim. Biophys. Acta 1864, 1185-1193, (2019)

### SUMMARY OF INVENTION

### Technical Problem

Nannochloropsis is an alga with higher lipid accumulation than other algae, and further technical development is desired for production cost reduction. The present invention has been made against such background, and an object of the present invention is to provide means for causing increase in the amounts of TAG accumulated in algae with high lipid accumulation such as Nannochloropsis.

### Solution to Problem

A previous report has demonstrated that, under phosphorus-deficient conditions, the amounts of lipid accumulated in an spx2 gene-disrupted strain is larger than those in the wild strain (International Publication No. WO 2020/050412). Strains with double-disruption of TGL1 and TGL2, homologs of the main TAG lipase in plants, are reported not to provide increased lipid production at practical levels (Biochim. Biophys. Acta 1864, 1185-1193, (2019)). With this in mind, the present inventors have diligently conducted a study to achieve the above object, and newly focused on class 3 lipase (Pfam #PF01764) as a TAG lipase that functions in surface layers of lipid droplets. Its structure represents a domain having an α/β hydrolase fold such as feruloyl esterase A of Aspergillus niger (J. Mol. Biol. 338, 495-506, (2004)), the triacylglycerol lipase OBL1 of Arabidopsis thaliana (New Phytol 217, 1062-1076, (2018)), and human diacylglycerol lipase a (Proc Natl Acad Sci USA 113, 26-33, (2016)). Pfam domain search found 23 class 3 lipases from Nannochloropsis. Then, for No3LIP7 and No3LIP14 among them, strains gene-disrupted for No3LIP7 and No3LIP14 by genome editing were produced from a strain whose SPX2 gene had been disrupted by genome editing as a parent strain. As a result, the spx2 No3lip7 and spx2 No3lip14 genes-double-disrupted strains successfully exhibited further increase in the amounts of lipid accumulated under phosphorus-deficient conditions.

The present invention provides the following [1] to [6].
[1] An alga having the following characteristics (1) and (2):
   (1) having reduced expression of a class 3 lipase gene; and
   (2) having reduced expression of an SPX2 gene.
[2] The alga according to [1], wherein the alga is an alga belonging to the genus Nannochloropsis.
[3] The alga according to [1], wherein the class 3 lipase gene is a gene encoding the following protein (a), (b), or (c):
   (a) a protein consisting of an amino acid sequence set forth in SEQ ID NO: 2, 4, 6, or 8;
   (b) a protein consisting of an amino acid sequence derived by substitution, addition, or deletion of 1 to 50 amino acid residues from an amino acid sequence set forth in SEQ ID NO: 2, 4, 6, or 8 and having lipase activity; and
   (c) a protein consisting of an amino acid sequence having a homology of 40% or more with an amino acid sequence set forth in SEQ ID NO: 2, 4, 6, or 8 and having lipase activity.
[4] The alga according to [1], wherein the SPX2 gene is a gene encoding the following protein (a), (b), or (c):
   (a) a protein consisting of an amino acid sequence set forth in SEQ ID NO: 10;
   (b) a protein consisting of an amino acid sequence derived by substitution, addition, or deletion of 1 to 50 amino acid residues from an amino acid sequence set forth in SEQ ID NO: 10; and
   (c) a protein consisting of an amino acid sequence having a homology of 60% or more with an amino acid sequence set forth in SEQ ID NO: 10, wherein the gene exhibits an increased expression level under phosphorus-deficient conditions.
[5] A method of producing triacylglycerol, comprising: culturing the alga according to any of [1] to [4] to allow the alga to produce triacylglycerol, and collecting the triacylglycerol produced.
[6] The method of producing triacylglycerol according to [5], wherein the culturing the alga occurs under phosphorus-deficient conditions.

### Advantageous Effects of Invention

The present invention provides a novel alga. This alga accumulates a large amount of TAG, and therefore is useful for production of TAG. Being free of any exogenous gene, the alga does not correspond to a recombinant organism, and thus allows outdoor culture.

### BRIEF DESCRIPTION OF DRAWINGS

FIG. 1 is a series of diagrams showing light/temperature conditions for culture. A. A light/temperature control program used for culture. Solid lines represent light intensity, and dashed lines represent temperature. B. Culture conditions for preculture and main culture.

FIG. 2 is a series of graphs for comparison of the wild strain, spx2 No3lip7 strain, and spx2 No3lip14 strain under phosphorus-deficient conditions. A. Variations of cell density in culture solution. B. Variations of amounts of biomass per cell. C. Variations of amounts of TAG accumulated per culture solution. D. Variations of amounts of TAG accumulated per biomass. E. Variations of amounts of TAG accumulated per cell. F. Variations of TAG accumulation efficiency.

FIG. 3 is a series of graphs for comparison of spx2 strain and spx2 No3lip14 strain under phosphorus-deficient conditions. A. Variations of cell density in culture solution. B. Variations of amounts of biomass per culture solution. C. Variations of amounts of biomass per cell. D. Variations of amounts of TAG accumulated per culture solution. E. Variations of amounts of TAG accumulated per cell. F. Variations of amounts of TAG accumulated per biomass. G. Variations of TAG accumulation efficiency. * indicates the presence of significant difference between spx2 strain and spx2 No3lip14 strain. n=4, error bar: se. p<0.05 (Tukey test).

FIG. 4 is a series of graphs for comparison of the growth of the wild strain, No3lip14 strain, spx2 strain, and spx2 No3lip14 strain under phosphorus-deficient conditions. A. Variations of cell density in culture solution. B. Variations of amounts of biomass per culture solution. C. Variations of amounts of biomass per cell. D. Variations of forward scattered light (FSC). E. Fluorescence micrographs (Nile Red and chlorophyll) and DIC micrographs of cells after culture.

FIG. 5 is a series of graphs for comparison of the amounts of TAG accumulated in the wild strain, No3lip14 strain, spx2 strain, and spx2 No3lip14 strain under phosphorus-deficient conditions. A. Variations of amounts of TAG accumulated per culture solution. B. Variations of amounts of TAG accumulated per biomass. C. Variations of amounts of TAG accumulated per cell. D. Variations of TAG accumulation efficiency.

### DESCRIPTION OF EMBODIMENTS

The following describes the present invention in detail.

The alga of the present invention is preferably an alga belonging to the genus Nannochloropsis, but may be any other alga. Examples of the algae belonging to the genus Nannochloropsis can include Nannochloropsis oceanica, Nannochloropsis gaditana, Nannochloropsis salina, Nannochloropsis oculata, Nannochloropsis atomus, Nannochloropsis maculata, Nannochloropsis granulata, Nannochloropsis limnetica, Nannochloropsis maritima, and Nannochloropsis australis.

The alga of the present invention has the following two characteristics.

The first characteristic is having reduced expression of a class 3 lipase gene. Class 3 lipase has a domain having an α/β hydrolase fold. Accordingly, the class 3 lipase gene in the alga can be identified on the basis of that domain (e.g., accession number in Pfam database: PF01764). The class 3 lipase gene is preferably for class 3 triacylglycerol lipase.

Specific examples of the class 3 lipase gene can include No3LIP7, No3LIP14, NO3LIP6, No3LIP10, and genes corresponding to those genes in individual algal species. The nucleotide sequences of No3LIP7, No3LIP14, No3LIP6, and No3LIP10 are as set forth in SEQ ID NOS: 1, 3, 5, and 7, respectively, and the amino acid sequences of proteins encoded by those genes are as set forth in SEQ ID NOs: 2, 4, 6, and 8, respectively. Examples of genes corresponding to No3LIP7, No3LIP14, NO3LIP6, or No3LIP10 can include a gene encoding (b) a protein consisting of an amino acid sequence derived by substitution, addition, or deletion of 1 to 50 amino acid residues from an amino acid sequence set forth in SEQ ID NO: 2, 4, 6, or 8 and having lipase activity, or (c) a protein consisting of an amino acid sequence having a homology of 40% or more with an amino acid sequence set forth in SEQ ID NO: 2, 4, 6, or 8 and having lipase activity.

The number of amino acid residues to be substituted, added, or deleted in the protein (b) may be any number of 1 to 50 without limitation, and can be preferably about 1 to 30, more preferably about 1 to 10, even more preferably about 1 to 5, and particularly preferably about 1, 2, 3, or 4.

The homology of the protein (c) with an amino acid sequence set forth in SEQ ID NO: 2, 4, 6, or 8 may be any value of 40% or more without limitation, and is preferably 50% or more, more preferably 60% or more, even more preferably 70% or more, and particularly preferably 80% or more. The value of homology may be further higher, and can be, for example, 90% or more, 95% or more, 97% or more, 98% or more, or 99% or more. The homology of an amino acid sequence can be calculated by using the program BLASTP provided by NCBI (National Center of Biotechnology Information).

Blast search conducted for the amino acid sequences set forth in SEQ ID NOS: 2, 4, 6, and 8 found the presence of a protein having a homology of approximately 70% with that of Nannochloropsis gaditana for SEQ ID NO: 2 (No3LIP7), the presence of a protein having a homology of approximately 40% with those of Nannochloropsis gaditana and Nannochloropsis salina for SEQ ID NO: 4 (No3LIP14), the presence of a protein having a homology of approximately 80% with those of Nannochloropsis gaditana and Nannochloropsis salina for SEQ ID NO: 6 (No3LIP6), and the presence of a protein having a homology of approximately 60% with that of Nannochloropsis gaditana and a homology of approximately 70% with that of Nannochloropsis salina for SEQ ID NO: 8 (No3LIP10). From this, genes encoding the protein (c) (genes that have a homology of 40% or more at amino acid levels) are expected to include genes corresponding to No3LIP7, No3LIP14, No3LIP6, and No3LIP10 in algae belonging to the genus Nannochloropsis.

While the alga of the present invention has reduced expression of a class 3 lipase gene, the expression “reduced expression of a class 3 lipase gene” means that the expression level of a class 3 lipase gene is lower than that in the wild strain, and a case without any expression of a class 3 lipase gene is also included in the meaning. The reduced expression may be caused by a procedure that affects the gene in the genome (e.g., gene modification by genome editing, mutation by radiation), or by a procedure that does not affect the gene in the genome (e.g., suppression of gene expression by an RNAi method or an antisense method).

Examples of modification of a class 3 lipase gene can include gene knockout (gene disruption), introduction of a mutation into a protein-coding region, and introduction of a mutation into an expression-controlling region.

The second characteristic is having reduced expression of an SPX2 gene. SPX2 genes are described in many documents (e.g., Liu et al., Open Biol. 2018-Jan 3; 8 (1): 170231, International Publication No. WO 2020/050412), and hence the SPX2 gene in the alga can be identified on the basis of those documents.

Specific examples of the SPX2 gene can include an SPX2 gene of Nannochloropsis NIES-2145 strain and genes corresponding to that gene in individual algal species. The nucleotide sequence of the SPX2 gene of Nannochloropsis NIES-2145 strain is as set forth in SEQ ID NO: 9. The amino acid sequence of a protein encoded by that gene is as set forth in SEQ ID NO: 10. Examples of genes corresponding to the SPX2 gene of Nannochloropsis NIES-2145 strain can include a gene that exhibits an increased expression level under phosphorus-deficient conditions, and encodes (b) a protein consisting of an amino acid sequence derived by substitution, addition, or deletion of 1 to 50 amino acid residues from an amino acid sequence set forth in SEQ ID NO: 10, or (c) a protein consisting of an amino acid sequence having a homology of 60% or more with an amino acid sequence set forth in SEQ ID NO: 10.

The number of amino acid residues to be substituted, added, or deleted in the protein (b) may be any number of 1 to 50 without limitation, and can be preferably about 1 to 30, more preferably about 1 to 10, even more preferably about 1 to 5, and particularly preferably about 1, 2, 3, or 4.

The homology of the protein (c) with an amino acid sequence set forth in SEQ ID NO: 10 may be any value of 60% or more without limitation, and is preferably 70% or more, more preferably 80% or more, even more preferably 90% or more, and particularly preferably 95% or more. The value of homology may be further higher, and can be, for example, 97% or more, 98% or more, or 99% or more.

Whether a gene exhibits an increased expression level under phosphorus-deficient conditions can be determined in such a manner that an alga containing the gene is cultured under normal conditions (under conditions without phosphorus deficiency) and under phosphorus-deficient conditions, and the expression levels of the gene under the two conditions are compared.

The expression “reduced expression of an SPX2 gene” means that, as with the case of the class 3 lipase, the expression level of an SPX2 gene is lower than that in the wild strain, and a case without any expression of an SPX2 gene is also included in the meaning. The reduced expression may be caused by a procedure that affects the gene in the genome (e.g., gene modification by genome editing), or by a procedure that does not affect the gene in the genome (e.g., suppression of gene expression by an RNAi method or an antisense method). Examples of modification of an SPX2 gene can include gene knockout (gene disruption), introduction of a mutation into a protein-coding region, and introduction of a mutation into an expression-controlling region.

The method of producing TAG of the present invention comprises: culturing the alga of the present invention to allow the alga to produce TAG, and collecting the TAG produced.

The alga of the present invention may be cultured under normal conditions (under conditions without phosphorus deficiency), but is preferably cultured under phosphorus-deficient conditions to increase the amount of TAG accumulated. For matters other than phosphorus, appropriate culture conditions fitting with the type of the alga can be selected. For example, in the case that the alga to be cultured is an alga belonging to the genus Nannochloropsis, the medium to be used can be F2N medium, HD medium, or a medium obtained by removing phosphorus from any of the two, the culture temperature can be about 15 to 25° C., and the light intensity during culture can be 10 to 1000 μmol photons/m²/sec.

Examples of methods for collecting TAG produced in the alga can include methods that are conventionally used in recovering TAG accumulated in cells such as a method of recovering TAG by drying, freezing, crushing, filtering, centrifugation, or solvent extraction of algal cells.

### EXAMPLES

The following describes the present invention in more detail with examples, but the present invention is not limited to those examples.

### Experimental Materials

The eustigmatophyte Nannochloropsis NIES-2145 (hereinafter, referred to as “N. 2145”) was used. This algal strain is available from National Institute for Environmental Studies, Japan (http://www.nies.go.jp/).

### Genes Used

The transcript data of N. oceanica CCMP1779 v2.0 (https://phycocosm.jgi.doe.gov/Nanoce1779_2/Nanoce1779_2.home.html) was searched for novel lipase genes having the Lipase class 3 domain (PF01764), and 23 genes were selected as candidates. From the expression profiles of them, No3LIP7 and No3LIP14 were picked up. In addition, an SPX2 gene, which is a gene that exhibits an increased expression level under phosphorus-deficient conditions (International Publication No. WO 2020/050412), was used.

The sequences of the genes are listed in a sequence listing. The sequences of No3LIP7, No3LIP14, and SPX2 are set forth in SEQ ID NOS: 1, 3, and 9, respectively.

### Experimental Operations

### 1. Culture Conditions

HD medium was used as a medium for normal solution culture in culture of N.2145.

In 1 L of ion-exchanged water, 222 mg of ZnSO₄·7H₂O, 79 mg of CuSO₄·5H₂O, 15 mg of MoO₃, 2.86 g of H₃BO₃, and 1.81 g of MnCl₂·4H₂O were dissolved, and the resultant was stored in advance as A-5 stock at 4° C. A product obtained by dissolving 2.5 g of KNO₃, 0.25 g of Na₂HPO₄, 0.075 g of Fe-EDTA, and 5 mL of A-5 stock in 500 mL of ion-exchanged water and a product obtained by dissolving Daigo's Artificial Seawater SP (FUJIFILM Wako Pure Chemical Corporation) in 500 mL of ion-exchanged water were each sterilized in an autoclave and then mixed together, to which 2 mL of vitamin mix solution was added, and the resultant was used as HD medium. The vitamin mix solution had been prepared by dissolving 0.6 μg of Vitamin B₁₂, 0.3 μg of Biotin, and 60 μg of Thiamine HCl in 10 mL of ion-exchanged water, and filter-sterilized before use. HD medium removed of Na₂HPO₄ was used as phosphorus-deficient medium, and HD medium with the amount of Na₂HPO₄ reduced to 0.125 g was used as phosphorus 1/2 medium. In normal culture, 5 mL of culture solution cultured for 7 days was added to 50 mL of HD medium, and culture was performed under conditions of 700 μmol photons/m²/sec, 25° C., and aeration with 2% CO₂ at 15 mL/min. The flow cytometer RF-500 (SYSMEX CORPORATION) was used for counting the numbers of cells.

### 2. Method for Creating Spx2-Disrupted Strain and Lipase-Disrupted Strain

For gene disruption of the class 3 lipase genes No3LIP14 and NoLIP7 and the phosphate metabolic pathway gene SPX2, a genome editing tool called transcriptional activator-like effector (TALE) nucleases (TALENs) was used. A TALEN is an artificial nuclease obtained by fusing a TALE domain, which is a DNA-binding protein derived from a plant pathogen that enables free design of a target, and a FokI nuclease domain derived from a marine bacterium. The TALE domain has 16 to 18 sequences each called a TALE repeat and consisting of 34 amino acids, and one TALE repeat identifies one nucleotide of DNA. One DNA double-strand break can be introduced into a target sequence sandwiched between two TALENs: L-TALEN and R-TALEN. In the present experiment, a Platinum TALEN (PtTALEN) system, produced by modifying the amino acids in the fourth and 32nd TALE repeats to give further enhanced activity, was used. For PtTALEN target sequences, TALENs were picked up by using a web tool called TAL Effector Nucleotide Targeter 2.0 (https://tale-nt.cac.cornell.edu), the activities thereof were examined with cultured cells, and all-in-one PtTALEN vectors for expression in Nannochloropsis were constructed for TALENs from which high activity was found to use for mutation of the target genes. For PtTALENS for SPX2 disruption, two PtTALEN pairs were found to have mutation activity, and hence each used to construct an all-in-one PtTALEN vector for expression in Nannochloropsis, the vector was introduced into Nannochloropsis, and sequences around the target were PCR-amplified to examine mutations by direct sequencing. The target sequences were SPX A (L-TALEN: TGCTAGAAGCCGCACCGC, spacer: TAGCATCATGAAGTT, R-TALEN: TGGTCTGTATCTGCGCGA) and SPX D (L-TALEN: TGCAATACGACAAACTAA, spacer: AGCGAATGATTCGCA, R-TALEN: TTCTGGCCGAAGTGGAGA), and 4-11-3 strain (7 nucleotides deleted) was obtained by using an all-in-one PtTALEN vector targeting SPX A, 5-9-2 strain (5 nucleotides deleted) and 5-19-2 strain (4 nucleotides inserted) were obtained by using an all-in-one PtTALEN vector targeting SPX D, and 8-4-1 strain (82 nucleotides deleted) was further obtained by simultaneously introducing an all-in-one PtTALEN vector targeting SPX A and an all-in-one PtTALEN vector targeting SPX D; thus, four SPX2 frameshift mutants were obtained in total.

Further, double-disrupted strains were constructed for the genes. An all-in-one PtTALEN vector targeting LIP7 D (L-TALEN: TTCCAGCAGCAGCCAGTA spacer: TCATCGTCGCTCACC R-TALEN: ACCAAGCCCGCAGCACCA), from which gene disruption ability had been previously detected, was introduced into 8-4-1 strain, which is an SPX2-disrupted strain; thereby, two SPX2/No3LIP7-double-disrupted strains, 2-4-4-1 strain (SPX2: 82 nucleotides deleted, No3LIP7: 2 nucleotides deleted) and 2-8-5-1 strain (SPX2: 82 nucleotides deleted, No3LIP7: 1 nucleotide inserted), were obtained. In addition to this, an all-in-one PtTALEN vector targeting LIP14 A (L-TALEN: TCAGTCTGCGGCATGCCC spacer: TTGTGTCGGGCGCGC L-R-TALEN: CAGCCGCCGTGGCTGCGA), from which mutation activity had been previously detected, was introduced into 8-4-1 strain, similarly; thereby, two SPX2/No3LIP14-double-disrupted strains, 13-12 strain (SPX2: 82 nucleotides deleted, No3LIP14: 7 nucleotides deleted) and 13-15 strain (SPX2: 82 nucleotides deleted, No3LIP14: 10 nucleotides deleted), were obtained.

In addition to the above, 6B-6 strain (No3LIP14: 13 nucleotides deleted) and 6B-10 strain (No3LIP14: 17 nucleotides deleted), in each of which only a class 3 lipase gene had been disrupted, were constructed. The construction of the strains with single disruption of a class 3 lipase gene was performed in the same manner as the construction method for the other double-disrupted strains except that 8-4-1 strain was replaced with the wild strain. The sequences used for mutation are set forth in SEQ ID NOS: 11 to 22 in the sequence listing.

### 3. Culture Under Phosphorus-Deficient Conditions

In Experiment 1, cells of each of the wild strain, 2-4-4-1 strain (SPX2: 82 nucleotides deleted, No3LIP7: 2 nucleotides deleted), and 13-12 strain (SPX2: 82 nucleotides deleted, No3LIP14: 7 nucleotides deleted) subjected to normal culture for 7 days were successively seeded in 500 mL of HD medium to 1×10⁸ cells/mL, and cultured under 16-hour light period (700 μmol photons/m²/sec, one side irradiated) and 8-hour dark period at 25° C. for 4 days, while aeration was performed with 2% CO₂ at 300 mL/min. Thereafter, the cells were successively seeded on 500 mL in phosphorus-deficient medium to 1×10⁸ cells/mL, and cultured under 16-hour light period (500 μmol photons/m²/sec, both sides irradiated, 22° C.) and 8-hour dark period (25° C.) for 2 days, and under 16-hour light period (1000 μmol photons/m²/sec, both sides irradiated, 15° C.) and 8-hour dark period (25° C.) for 10 days, while aeration was performed with 2% CO₂ at 450 mL/min.

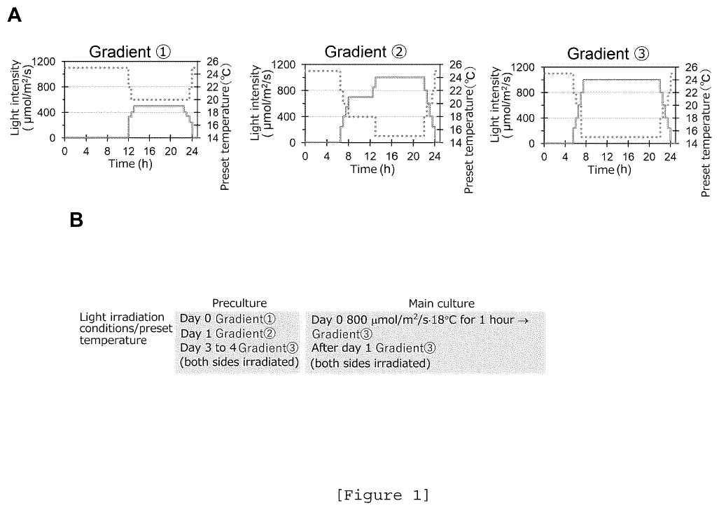

In Experiment 2, cells of each of 8-4-1 strain (SPX2: 82 nucleotides deleted) and 13-12 strain (SPX2: 82 nucleotides deleted, No3LIP14: 7 nucleotides deleted) subjected to normal culture for 7 days were successively seeded in 500 mL of phosphorus 1/2 medium to 1×10⁸ cells/mL, and cultured under the light irradiation conditions at the preset temperature of the incubator as shown in FIG. 1A for 4 days, while aeration was performed with 2% CO₂ at 450 mL/min. Thereafter, the cells were successively seeded in 500 mL of phosphorus-deficient medium to 2× 10⁸ cells/mL, giving four samples in each case, and cultured under the light irradiation conditions at the preset temperature of the incubator as shown in FIG. 1A for 10 days, while aeration was performed with 2% CO₂ at 450 mL/min.

In Experiment 3, cells of each of the wild strain, 6B-6 strain (No3LIP14: 13 nucleotides deleted), 8-4-1 strain (SPX2: 82 nucleotides deleted), and 13-12 strain (SPX2: 82 nucleotides deleted, No3LIP14: 7 nucleotides deleted) subjected to normal culture for 7 days were successively seeded in 500 mL of phosphorus 1/2 medium to 1×10⁸ cells/mL, and cultured under the light irradiation conditions at the preset temperature of the incubator as shown in FIG. 1A for 4 days, while aeration was performed with 2% CO₂ at 450 mL/min. Thereafter, the cells were successively seeded in 500 mL of phosphorus-deficient medium to 2× 10⁸ cells/mL, giving four samples in each case, and cultured under the light irradiation conditions at the preset temperature of the incubator as shown in FIG. 1A for 10 days, while aeration was performed with 2% CO₂ at 450 mL/min.

### 4. Lipid Extraction

On each day, 10 mL of culture solution was collected, and centrifuged at 4670 G and 25° C. for 10 minutes to precipitate cultured cells. After the centrifugation, the supernatant was removed, and the precipitated cultured cells were rapidly frozen with liquid nitrogen, and stored at −80° C.

The frozen cells were suspended in 0.8 mL of ion-exchanged water, 1 mL of chloroform and 2 mL of methanol were added, and the resultant was stirred and left to stand at room temperature for 1 hour. Thereto, 1 mL of chloroform and 1 mL of ion-exchanged water were added to form a suspension, which was centrifuged with a swing rotor at 1000×g for 5 minutes, and the water/methanol layer (upper layer) was removed and the chloroform layer (lower layer) was transferred to an additional glass test tube. Meanwhile, 1.5 mL of chloroform was added to the original glass test tube to form a suspension. This original test tube with the suspension and the additional test tube to which the chloroform layer had been transferred were together centrifuged with a swing rotor at 1000×g for 5 minutes. After the centrifugation, the chloroform layer in the additional test tube was transferred to another test tube the weight of which had been measured. The chloroform layer in the original test tube was recovered, centrifuged with a swing rotor at 1000×g for 5 minutes and recovered, and combined with the previous chloroform extract to give a lipid extract. This lipid extract was dried with a vacuum concentrator, dissolved in chloroform to 10 mg/mL, and then stored at −20° C.

### 5. Lipid Analysis

Lipid extract was spotted on a thin-layer silica plate, and developed with developing solution of 160 mL of hexane, 40 mL of diethyl ether, and 4 mL of acetic acid for 35 minutes. TAG was confirmed with 0.001% (w/v) primuline under UV irradiation. Silica in the part having TAG thereon was scraped, and 10 μL of 5 mM heneicosanoic acid and 2.5 mL of 1.5 M hydrochloric acid/methanol were added to form a suspension, which was then left to stand at 85° C. for 2.5 hours for methyl-esterification of fatty acids. Thereto, 2.5 mL of hexane was added to form a suspension, which was then left to stand, and methyl-esterified fatty acids in the upper layer were recovered. The methyl-esterified fatty acids recovered were dried, and then dissolved in 100 μL of hexane, giving a gas chromatography sample. Gas chromatography was performed by using a SHIMADZU GC-2014 equipped with an HR-SS-10 (0.25φ×25 m) (SHINWA CHEMICAL INDUSTRIES, LTD.).

### 6. Measurement of Biomass (Dry Weight of Cells)

On each day, 10 mL of culture solution cultured with phosphorus-deficient medium was collected, transferred to a 50-mL tube, and centrifuged at 4670 G and 25° C. for 10 minutes. After the centrifugation, the supernatant was removed with care not to remove any cell. The residual precipitate was suspended by adding H₂O, and the suspension was transferred to a 1.5-mL tube the weight of which had been measured. Centrifugation was performed at 7000 G and 25° C. for 10 minutes. The supernatant was removed with care not to remove any cell, and the 1.5-mL tube was placed in a high-temperature dryer, and dried at 105° C. for 5 hours with the cap removed. The 1.5-mL tube was taken out of the high-temperature dryer, and the biomass was weighed with an electronic balance.

### Experimental Results

### 1. Experiment 1: Comparison of Wild Strain, Spx2 No3Lip7 Strain, and Spx2 No3Lip14 Strain Under Phosphorus-Deficient Conditions

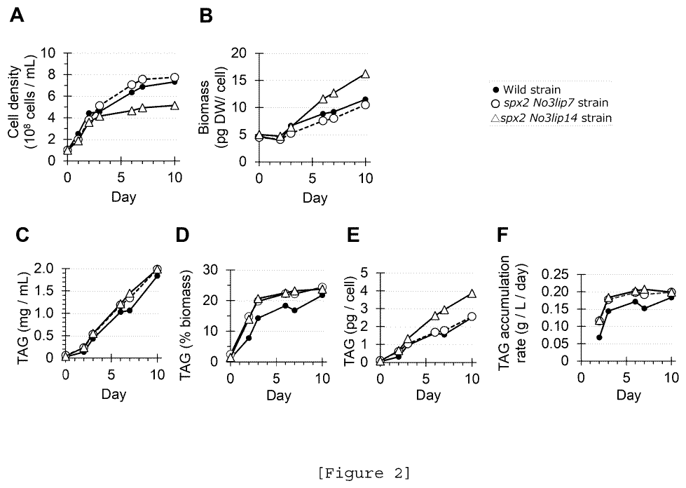

As shown in FIG. 2A, the cell density of spx2 No3lip7 strain was slightly higher than that of the wild strain on day 3, 6, 7, and 10 of culture under phosphorus-deficient conditions. The cell density of spx2 No3lip14 strain was lower than that of the wild strain through the whole culture period. As shown in FIG. 2B, on day 6, 7, and 10 of culture, the amounts of biomass of spx2 No3lip14 strain per cell were larger than those of the wild strain, and the amounts of biomass of spx2 No3lip7 strain were slightly smaller than those of the wild strain. As shown in FIGS. 2C, 2D, and 2F, the amounts of TAG accumulated per culture solution and per biomass and TAG accumulation efficiency in spx2 No3lip7 strain and spx2 No3lip14 strain were larger/higher than those in the wild strain. As shown in FIG. 2E, the amounts of TAG accumulated per cell in spx2 No3lip7 strain were comparable to those in the wild strain, but the amounts of TAG accumulated per cell in spx2 No3lip14 strain were larger than those in the wild strain.

### 2. Experiment 2: Comparison of Spx2 Strain and Spx2 No3Lip14 Strain Under Phosphorus-Deficient Conditions

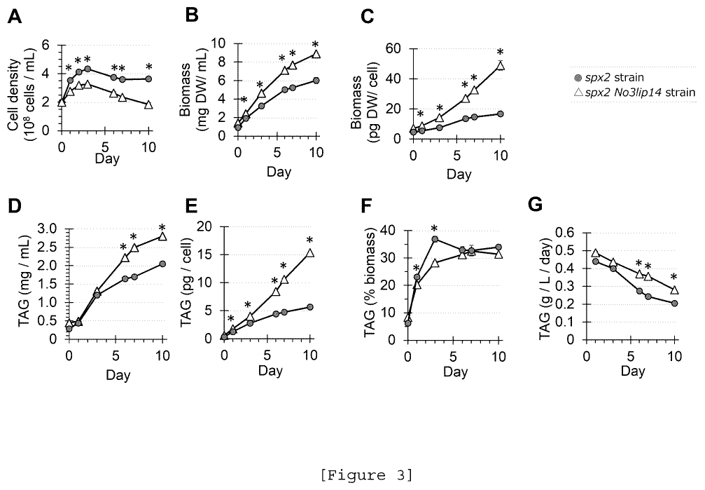

As shown in FIG. 3, under phosphorus-deficient conditions, the cell density of spx2 No3lip14 strain was significantly lower than that of spx2 strain. The amounts of biomass per culture solution and per cell in spx2 No3lip14 strain were significantly larger than those of spx2 strain after day 1 of culture. The amounts of TAG accumulated per culture solution and the amounts of TAG accumulated per cell in spx2 No3lip14 strain were significantly higher than those in spx2 strain after day 6 of culture and after day 1 of culture, respectively. The amounts of TAG accumulated per biomass in spx2 No3lip14 strain were significantly smaller than those in spx2 strain on day 1 and 3 of culture, but there was no difference after day 6. The TAG accumulation efficiency in spx2 No3lip14 strain was significantly higher than that in spx2 strain after day 6 of culture.

### 3. Experiment 3: Comparison of Wild Strain, No3Lip14 Strain, Spx2 Strain, and Spx2 No3Lip14 Strain Under Phosphorus-Deficient Conditions

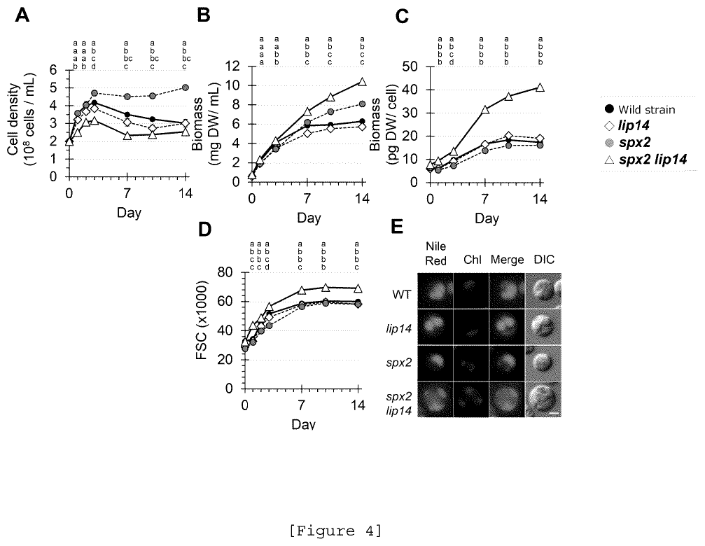

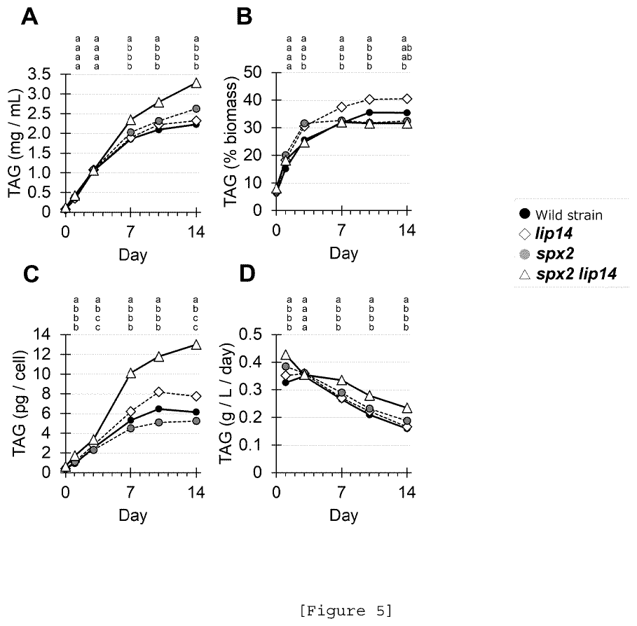

As shown in FIG. 4A, under phosphorus-deficient conditions, the cell density of spx2 No3lip14 strain was lower than those of the wild strain, No3lip14 strain, and spx2 strain, but, as shown in FIGS. 4B and 4C, the amounts of biomass per culture solution and per cell were larger in spx2 No3lip14 strain. Further, as shown in FIG. 4D, the values of forward scattered light for cells of spx2 No3lip14 strain as measured with a flow cytometer were higher than those for the wild strain, No3lip14 strain, and spx2 strain, suggesting that the cell size of spx2 No3lip14 strain was larger than those of the other strains, and microscopic observation found cells of large diameter at high frequency. As shown in FIGS. 5A, 5C, and 5D, the amounts of TAG accumulated per culture solution and per cell and TAG accumulation efficiency were all higher in spx2 No3lip14 strain than those in the wild strain, No3lip14 strain, and spx2 strain.

### 4. Summary

The above results revealed that the strains having the SPX2 gene and a novel lipase gene (No3LIP7 or No3LIP14) disrupted by genome editing had larger amounts of TAG accumulated under phosphorus-deficient conditions than the wild strain. In particular, spx2 No3lip14 strain had increase in the amounts of biomass and amounts of TAG accumulated per cell, and therefore is suitable for TAG production under phosphorus-deficient conditions. As compared with the strain with spx2 single genome editing, spx2 No3lip14 strain similarly exhibited enhancement in the amounts of biomass and amounts of TAG accumulated per culture solution and per cell, and it was demonstrated that disruption of both the SPX2 gene and the lipase gene No3LIP14 by genome editing results in higher TAG accumulation ability than single disruption of the SPX2 gene.

### INDUSTRIAL APPLICABILITY

The present invention is applicable to industrial fields relating to fuels and others.

1. An alga having the following characteristics (1) and (2):
(1) having reduced expression of a class 3 lipase gene; and
(2) having reduced expression of an SPX2 gene.

2. The alga according to claim 1, wherein the alga is an alga belonging to the genus Nannochloropsis.

3. The alga according to claim 1, wherein the class 3 lipase gene is a gene encoding the following protein (a), (b), or (c):
(a) a protein consisting of an amino acid sequence set forth in SEQ ID NO: 2, 4, 6, or 8;
(b) a protein consisting of an amino acid sequence derived by substitution, addition, or deletion of 1 to 50 amino acid residues from an amino acid sequence set forth in SEQ ID NO: 2, 4, 6, or 8 and having lipase activity; and
(c) a protein consisting of an amino acid sequence having a homology of 40% or more with an amino acid sequence set forth in SEQ ID NO: 2, 4, 6, or 8 and having lipase activity.

4. The alga according to claim 1, wherein the SPX2 gene is a gene encoding the following protein (a), (b), or (c):
(a) a protein consisting of an amino acid sequence set forth in SEQ ID NO: 10;
(b) a protein consisting of an amino acid sequence derived by substitution, addition, or deletion of 1 to 50 amino acid residues from an amino acid sequence set forth in SEQ ID NO: 10; and
(c) a protein consisting of an amino acid sequence having a homology of 60% or more with an amino acid sequence set forth in SEQ ID NO: 10,
wherein the gene exhibits an increased expression level under phosphorus-deficient conditions.

5. A method of producing triacylglycerol, comprising: culturing the alga according to claim 1 to allow the alga to produce triacylglycerol, and collecting the triacylglycerol produced.

6. The method of producing triacylglycerol according to claim 5, wherein the culturing the alga occurs under phosphorus-deficient conditions.
